(19) United States
(12) Patent Application Publication
(10) Pub. No.: US 2013/0004959 A1
(43) Pub. Date: Jan. 3, 2013

(54) SCREENING DEVICE CONTAINING NANOG PROMOTER, SOX2 PROMOTER, LIN 28 PROMOTER, OCT4 PROMOTER AND LUCIFERASE GENE AND SCREENING METHOD THEREOF

(75) Inventors: Shen Laing CHEN, Taoyuan County (TW); Wei Jhen HUANG, Taoyuan County (TW); Moo Rung LOO, Taoyuan County (TW)

(73) Assignee: NATIONAL CENTRAL UNIVERSITY, Taoyuan County (TW)

(21) Appl. No.: 13/342,347

(22) Filed: Jan. 3, 2012

(30) Foreign Application Priority Data
Jun. 29, 2011 (TW) 100122878

(51) Int. Cl. C12Q 1/68

(52) U.S. Cl. 435/6.13

(57) ABSTRACT

A screening device that contains Nanog promoter, Sox2 promoter, Lin28 promoter, Oct4 promoter and luciferase gene and a method thereof is revealed. The screening device is used to find out small molecules that improve activity of the promoters mentioned-above. A reporter plasmid formed by a pStable vector is introduced into stable somatic cells or stem cells with reporter plasmids such as a C2C12 mouse myoblast cell line and a P19 embryonal carcinoma cell line. By the luciferase gene contained in the pStable vector and pluripotent gene promoters placed upstream of the luciferase gene, luciferase expression is assayed by luminescence intensity measured. Thus response and activity of the promoters placed upstream of the luciferase gene is measured and small molecules inducing pluripotency are found out. If a drug can activate all promoters or respective promoter, it can be used as a drug that induces cell pluropatency.

### BACKGROUND OF THE INVENTION

1. Field of the Invention

The present invention relates to a screening device and a screening method thereof, especially to a screen device containing Nanog promoter, Sox2 promoter, Lin28 promoter, Oct4 promoter, and luciferase gene and a method thereof used for screening small-molecule compounds that increase activity of the above promoters so as to have more drugs available.

2. Description of Related Art

Embryonic stem cells are pluripotent cells that are able to differentiate into a plurality of tissues but these cells can't develop into an individual. The differentiation property allows embryonic stem cells to be employed as useful tools for both research and regenerative medicine. Many diseases could potentially be treated by pluripotent embryonic stem cells. However, the embryonic stem cells have a limited number and cell types. And the sources of the embryonic stem cells raise ethical issues. Besides the ethical concerns, the graft may lunch an immune response of the host's immune system and result in the graft rejection. Thus induced Pluripotent Stem Cells similar to natural pluripotent stem cells in many respects are important in research and clinical uses.

In 2006, mouse fibroblasts were induced into iPS cells by reprogramming technique and four key pluripotency genes essential for the production of pluripotent stem cells including Oct4, Sox2, Klf4 and c-Myc were isolated. In 2007, Thomson and colleagues have successfully transformed human fibroblasts into pluripotent stem cells using four transcription factors Oct4•Sox2•Lin28 and Nanog. Thomson et al. reported that c-Myc and Klf4 were unnecessary for generation of human iPS cells. The c-myc gene is a proto-oncogene implicated in cancer. Thus the cancer threat caused by the expression of oncogenes is reduced. However, problems such as mutational effects of virus insertion on genomes, reduced efficiency of reprogramming, etc. remain.

Although iPS cells overcome the problems of sources of embryonic stem cells and the graft rejection, there are still so many problems existed such as insertion mutagenesis and poor reprogramming efficiency. In recent years, many small molecules including VPA, BIX-01294, 5-aza-cytidine, etc. are reported to increase the reprogramming efficiency dramatically. One of the objects of the present invention is to find out the small-molecule compounds that improve cell pluripotency and help induction of iPS cells using cell line screening”.

In Nat Biotechnol 2008, Huangfu D. and colleagues have reported that an Oct4-GFP reporter gene can be used as an indicator of cell pluripotency. Compared with drug screening using Oct4-GFP cell line, the present invention uses Luciferase reporter assay to quantify the influence of the drug on the promoter activity for following statistics and comparisons. Thus whether the drug improves cell pluripotency is learned easily by using the screen device of the present invention. Moreover, the promoters of four transcription factors—Nanog, Sox2, Lin28, and Oct4 are used in the present invention for drug screening. If the drug can activate various genes at the same time or activate respective gene, it enhances cell pluripotency. Thus more drugs are available for improvement and maintenance of cell pluripotency.

### SUMMARY OF THE INVENTION

Therefore it is a primary object of the present invention to provide a screen device that contains Nanog promoter, Sox2 promoter, Lin28 promoter, Oct4 promoter and luciferase gene and a method thereof that finds out small-molecule compounds involved in improvement of cell pluripotency. The activity of the above promoters is also improved by the compounds.

It is another object of the present invention to provide a screening device that contains Nanog promoter, Sox2 promoter, Lin28 promoter, Oct4 promoter and luciferase gene and a method thereof that performs statistics and makes comparison according to expression of the luciferase gene during screening processes.

In order to achieve the above objects, a screening device containing Nanog promoter, Sox2 promoter, Lin28 promoter, Oct4 promoter and luciferase gene and a method of the same. The method reveals how to deliver the above promoters into pStable vector to form reporter plasmids. Then reporter plasmids are introduced into cells to produce the screen device. Next use a luminometer to measure the luminescence and quantify gene expression.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a schematic drawing showing a construction of an embodiment of a screening device according to the present invention;

FIG. 2 is a schematic drawing showing steps of an embodiment of a screening method according to the present invention;

FIG. 3 is a schematic drawing showing a pStable vector used as a vector backbone of an embodiment according to the present invention;

FIG. 4 is a schematic drawing showing pStable-Nanog-p-Luc plasmid of an embodiment according to the present invention;

FIG. 5 is a schematic drawing showing pStable-Sox2-p-Luc plasmid of an embodiment according to the present invention;

FIG. 6 is a schematic drawing showing an inner primer and an outer primer designed for preparing Sox2 primer of an embodiment according to the present invention;

FIG. 7 is a schematic drawing showing pStable-Lin28-p-Luc plasmid of an embodiment according to the present invention;

FIG. 8 is a schematic drawing showing a forward primer containing a KpnI cutting site and a reverse primer having a KpnI cutting site designed for preparing Lin28 primer of an embodiment according to the present invention.

### DETAILED DESCRIPTION OF THE PREFERRED EMBODIMENT

In order to make a screen device of the present invention detect more small-molecule compounds, a reporter plasmid constructed by four promoters together with a pStable vector is introduced into cells for expansion of screening, and performing analysis and statistics by luciferase gene.

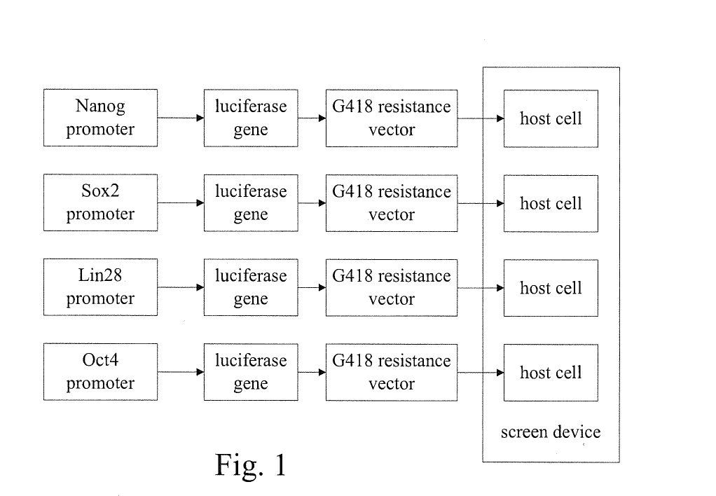

Refer to FIG. 1, in order to make a drug screening technique of the present invention also provide a screening of small-molecule compounds that induce formation of iPS cells, four reporter plasmids containing promoters of four transcription factors—Nanog, Sox2, Lin28, and Oct4, are individually introduced into different cells. After cell culture, transfection, and stable clone selection, a colony obtained is a stable clone in which a reporter plasmid gives stable expression.

A plasmid construction preparation method used to screen small molecules that increase activity of Nanog promoter, Sox2 promoter, Lin28 promoter, and Oct4 promoter includes following steps. Construct a reporter plasmid containing Nanog promoter, a luciferase gene, and a G418 resistant vector. Construct a reporter plasmid containing Sox2 promoter, a luciferase gene, and a G418 resistant vector. Construct a reporter plasmid containing Lin28 promoter, a luciferase gene, and a G418 resistant vector. Construct a reporter plasmid containing Oct4 promoter, a luciferase gene, and aG418 resistant vector. The above G418 resistant vector can be pStable vector.

Then these reporter plasmids are transfected into host cells. The host cell can be somatic cells, such as myoblast, or can be stem cells, such as embryonal carcinoma cells. Thus the host cell becomes a screening device for small-molecule compound screening. The small-molecule compounds can be histone deacetylase (HDAC), valporic acid (VPA), BIX (an inhibitor of the G9a histone methyltransferase), etc.

Moreover, the present invention further reveals another screening method that combines Nanog promoter, Sox2 promoter, Lin28 promoter, and Oct4 promoter with the luciferase gene and includes following steps. Firstly, construct a screening device contains Nanog promoter, Sox2 promoter, Lin28 promoter, Oct4 promoter and luciferase gene. Then the screening devices containing Nanog promoter, Sox2 promoter, Lin28 promoter, Oct4 promoter and the luciferase gene is added with an object under test. Next use a luminometer (luminescence reader) to detect and read luminescence of the screen device.

The screening is performed after the cells that express various promoters stably being prepared. The drug screening device of the present invention causes the substrate luciferin to emit light by means of the luciferase gene. Then the luminescence reader is used to detect luminescence and assess transcriptional activity of the promoter. At the same time, further statistics and analyses are performed according to measured results of the luminescence.

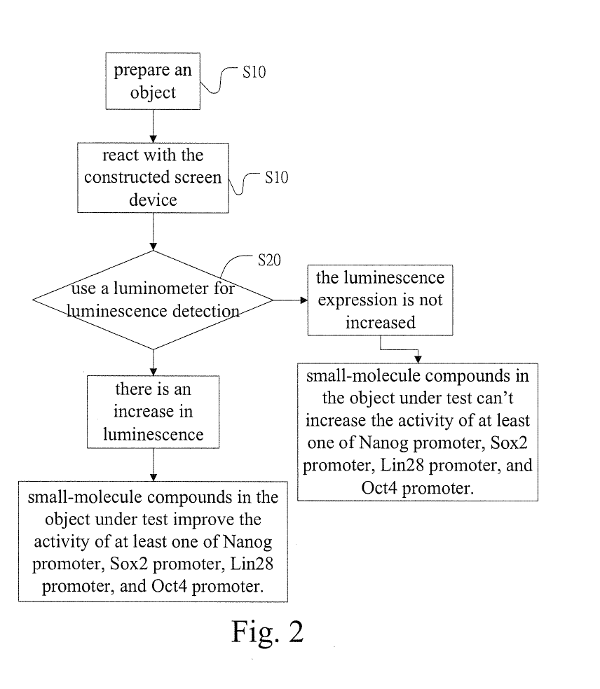

Refer to FIG. 2, a schematic drawing showing steps of an embodiment of a screening method according to the present invention are revealed. The method includes the following steps:
Step S10: prepare an object under test to react with the constructed screening device.
Step S20: use a luminometer for luminescence detection. If there is an increase in luminescence, this means small-molecule compounds in the object under test improve the activity of at least one of Nanog promoter, Sox2 promoter, Lin28 promoter, and Oct4 promoter. Thus the object can be applied to iPS cell induction for improving the induction efficiency. If the luminescence expression is not increased, this means small-molecule compounds in the object under test can't increase the activity of at least one of Nanog promoter, Sox2 promoter, Lin28 promoter, and Oct4 promoter.

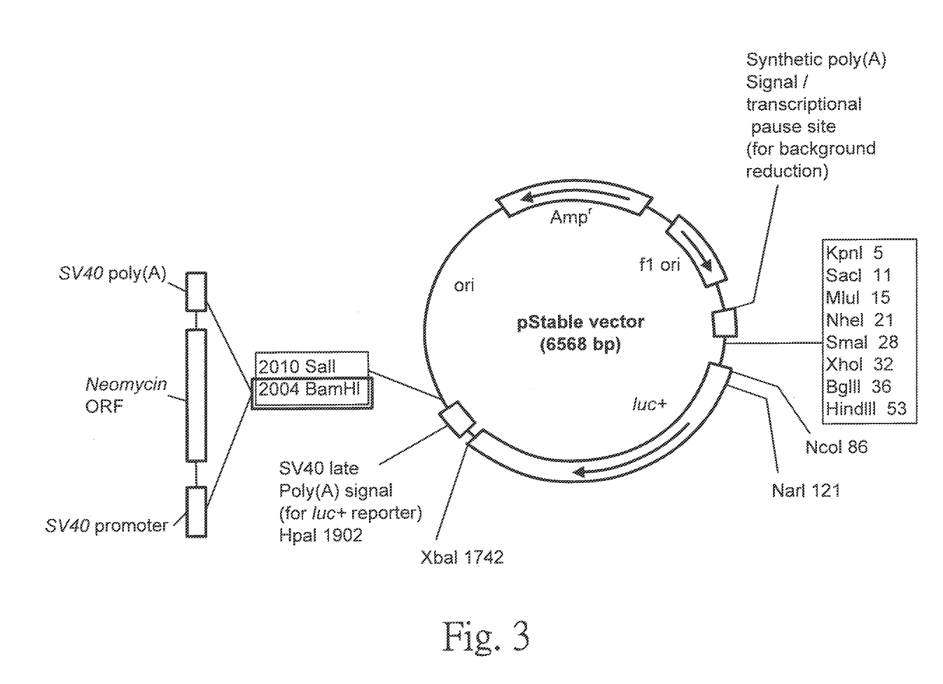

In an embodiment of the present invention, four reporter plasmids derived from a pStable vector and each contains one of the four promoters, Nanog promoter, Sox2 promoter, Lin28 promoter, and Oct4 promoter. The four promoters are respectively constructed and combined with both luciferase gene and G-418 resistance gene. The backbone of the reporter plasmid is the pStable vector. Refer to FIG. 3, a pStable vector used as a vector backbone in an embodiment of the present invention is revealed. The backbone of the reporter plasmid is constructed by following steps.

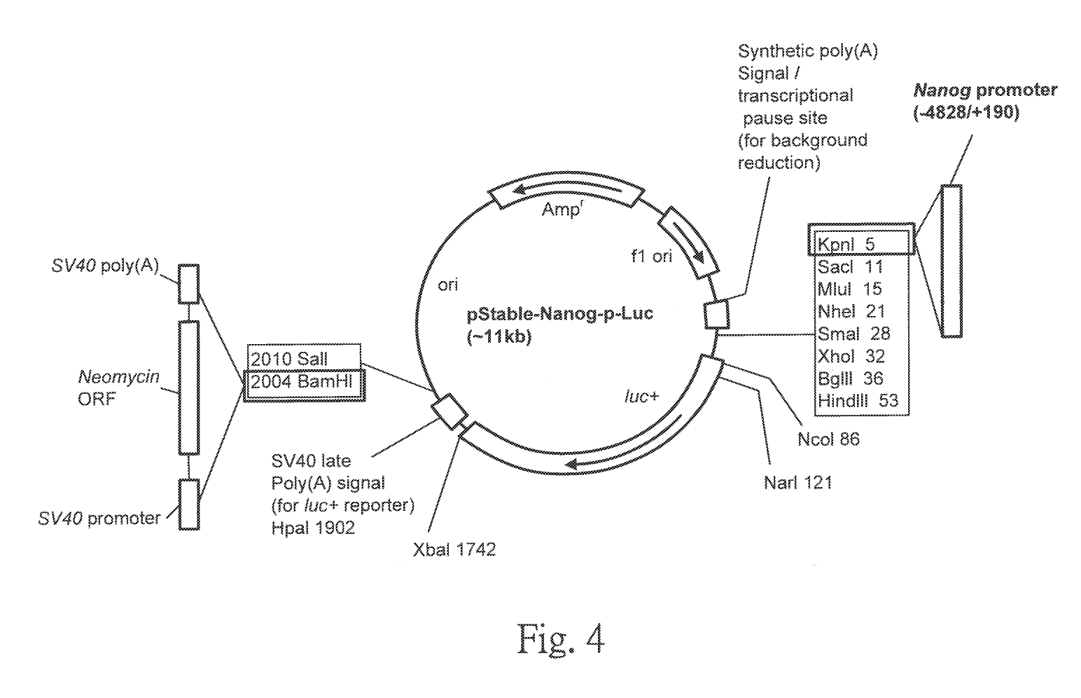

Firstly, construct a plasmid backbone that contains Nanog promoter, luciferase gene, and G418-resistance gene. The Nanog is one of key transcription factors involved in drug screening of the present invention. Refer to FIG. 4, a pStable-Nanog-p-Luc plasmid of an embodiment is revealed. As shown in figure, Nanog promoter in the plasmid Nanog 4.8 kb/pGL3 is cut by using KpnI and XhoI restriction enzymes and then is purified by gel extraction. After agarose gel electrophoresis of DNA, required DNA segment is cut from the gel and put into 1.5 ml microcentrifuge tube for purification.

The gel is added with equal-volume DF buffer and set in a 75 degrees Celsius water bath for 10 minutes so as to dissolve the gel. Then set at the room temperature for 5 minutes, the sample is added into a Kit. Pour the filtrate after being centrifuged at 13000 rpm for 1 minute.

Next 600 ml wash buffer is added. After being centrifuged at 13000 rpm for 3 minutes, the filtrate is removed. Then add buffer, centrifuge at 13000 rpm for 5 minutes and pour the filtrate again. The column is put into the 1.5 ml microcentrifuge tube, add 50 ml sterile water into the tube and set at room temperature for 1 hour or at 4 degrees Celsius overnight. Utilize gel electrophorisis for confirmation of the promoter.

Use restriction enzyme XhoI or KpnI to cut a certain amount of p Stable vector. Then the vector reacts with calf intestinal alkaline phosphatase (CIP) at 37° C. for 1 hour for removing 5′-phosphate groups of the vector and preventing self-ligation of the vector. The purified Nanog promoter, as an insert, is inserted into the pStable vector by T4 DNA ligase. After plasmid transformation and mini-preparation of plasmid DNA, pStable-Nanog-p-Luc plasmid is obtained.

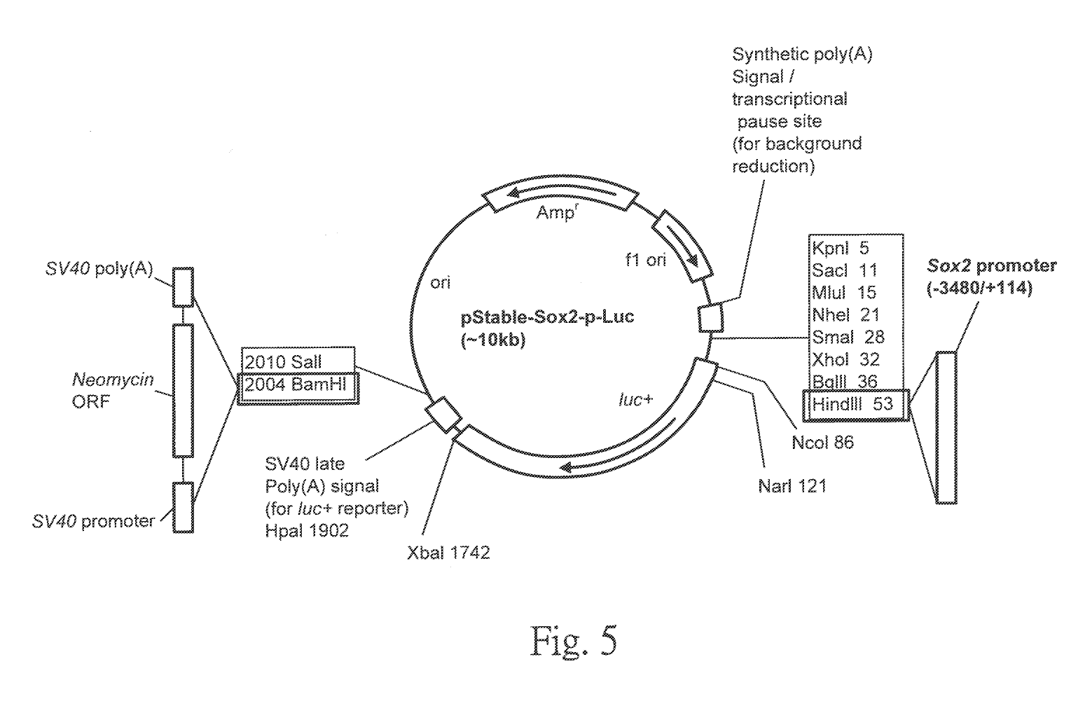

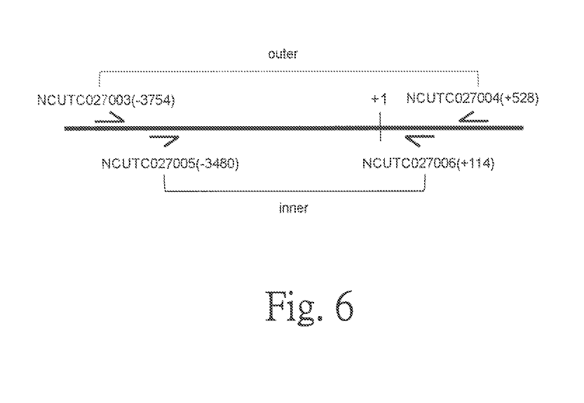

Secondly, construct a plasmid backbone that contains Sox2 promoter, luciferase gene, and G418-resistance gene. The Sox2 is one of key transcription factors involved in drug screening of the present invention. Refer to FIG. 5, a pStable-Sox2-p-Luc plasmid of an embodiment is revealed. A set of outer forward & reverse primer is designed. Then use Taq polymerase to get Sox2 promoter from mouse genome. Next an inner primer is further designed to amplify Sox2 promoter and use as an insert. As shown FIG. 6, a set of inner primer and a set of outer primer designed for preparing Sox2 primer of an embodiment are revealed.

Use the insert prepared by PCR (polymerase chain reaction) to produce a yT&A-Sox2-p plasmid after ligation, plasmid transformation and mini-preparation of plasmid DNA.

The Sox2 promoter of yT&A-Sox2-p plasmid is ligated into the pStable vector by a second construction. Thus the construction of the plasmid backbone contains luciferase gene and G418-resistance gene is completed.

Use restriction enzyme HindIII to release Sox2 promoter from yT&A-Sox2-p plasmid and prepare an insert. Also use restriction enzyme HindIII to cut pStable vector and remove 5′-phosphate groups of pStable vector by CIP.

Next perform ligation, plasmid transformation and mini-preparation of plasmid DNA by using the prepared vector and the prepared insert mentioned-above so as to complete the construction of pStable-Sox2-p-Luc plasmid.

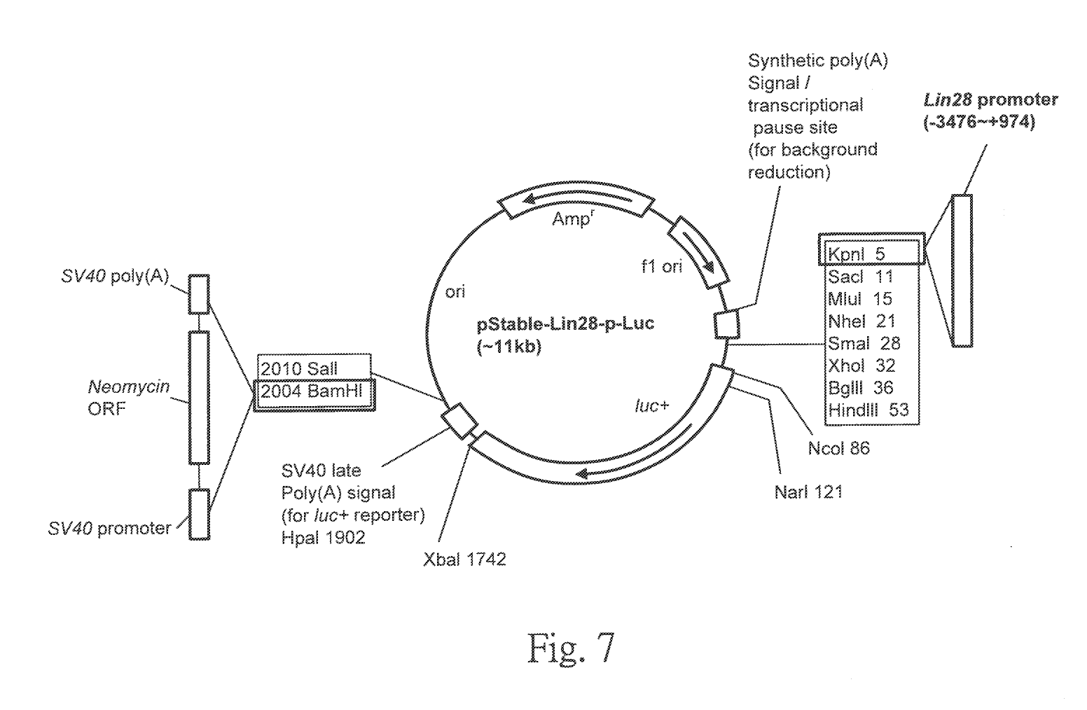

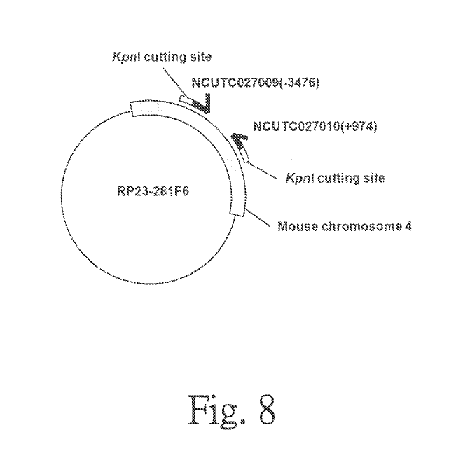

Furthermore, construct a plasmid backbone that contains Lin28 promoter, luciferase gene, and G418-resistance gene. The Lin28 is one of key transcription factors involved in drug screening of the present invention. Refer to FIG. 7, a pStable-Lin28-p-Luc plasmid of an embodiment is revealed. As shown in the figure, a forward primer and a reverse primer, both including a KpnI cutting site, are designed. Refer to FIG. 8, an embodiment of the present invention for preparing Lin28 promoter is revealed. The embodiment includes a forward primer containing a KpnI cutting site and a reverse primer having KpnI cutting site. Lin28 promoter on mouse BAC clone (RP23-281F6) is obtained by PCR and then cut by restriction enzyme KpnI for preparing an insert. Also use restriction enzyme KpnI to cut pStable vector and remove 5′-phosphate groups of pStable vector by CIP. Then perform ligation, plasmid transformation and mini-preparation of plasmid DNA by using the prepared vector and the prepared insert mentioned-above so as to get the pStable-Lin28-p-Luc plasmid.

At last, construct a plasmid backbone that contains Oct4 promoter, luciferase gene, and G418-resistance gene. The Oct4 is one of key transcription factors involved in drug screening of the present invention. Use restriction enzyme MluI and KpnI to release Oct4 proximal promoter from GOF-18 plasmid (the sequence—1888˜+44) and used as an insert. pStable vector is treated by restriction enzyme KpnI. Finally, pStable-Oct4-p-Luc plasmid is obtained by sticky end ligation with the insert and the vector.

The reporter plasmids whose backbone is constructed by the method mentioned above are transinfected individually into different dishes of host cells. After the reporter plasmids have been introduced into C2C12 or P19 cells, use G418 (Geneticin), a selective antibiotic for Neomycin resistance gene, to screen positive clones because pStable clones carry Neomycin resistance gene. After cell culture, transinfection, stable clone selection, and cells growing as colonies, stable clones in which various different promoters are expressed are obtained.

In an embodiment of the present invention, pStable-Nanog-p-Luc plasmid, pStable-Sox2-p-Luc plasmid, pStable-Lin28-p-Luc plasmid and pStable-Oct4-p-Luc plasmid are introduced into both a C2C12 cell line and a P19 cell line. C2C12 is a mouse myoblast cell line obtained through serial passage of myoblasts cultured from mouse muscle and can differentiate into myotube. P19 is an epithelial cell, isolated from mouse embryonal carcinoma cell (EC cell). The C2C12 cell and P19 cell are cultured in an incubator under normal growth conditions (typically at 37° C., 5% CO₂, and saturated humidity). Culture conditions of C2C12 cells and P19 cells are listed in the following list one:

### List One:

| | Cell culture environment |
|---|---|
| C2C12 | DMEM and 20% FBS |
| P19 | alpha-MEM and 10% FBS |

FBS: Fetal Bovine Serum (GIBCO, U.S.A)
DMEM: Dulbecco's Modified Eagle's Medium (JRH biosciences)

After the cell culture mentioned above, transfection is performed. Now seed C2C12 cells and P19 cells respectively onto different culture dishes. When cells grow over the surface of the culture dish, add prepared medium Mix I and medium Mix II into a 1.5 ml microcentrifuge tube. The medium components are listed in the list two.

### List Two:

| | Hepes | DNA | ddH₂O | lipofectamine |
|---|---|---|---|---|
| Mix I | 25 μl | 10 μl | 15 μl | — |
| Mix II | 25 μl | — | 12.5 μl | 12.5 μl |

Mix the medium Mix I with the medium Mix II and let stand at room temperature for 15˜30 minutes. Then the mixture is mixed with 900 ml serum free medium and added into the culture dish with cells. Fill more serum free medium into the culture dish up to the required volume. Next the culture dish with cells is incubated at 37° C. and 5% CO₂ for 24 hours. Change the medium to the original culture medium.

After the transfection processes, perform stable clone selection. The transfected cells are cultured in a culture medium containing 800 ug/ml G418. Replace the medium with fresh medium also containing antibiotics every two to three days for stable clone selection. Thus colonies obtained are stable clones in which various promoters give stable expression.

According to the luciferase expression measured by the luminometer, a screening device of the present invention is used to find out small-molecule compounds that improve activity of Nanog promoter, Sox2 promoter, Lin28 promoter, and Oct4 promoter. And use these small-molecule compounds to increase the promoter activity.

In an embodiment of the present invention, harvest cells after cells being reacted with 2 mM VPA for 24 hours and measure luminescence expression of cells. Moreover, retinoic acid (RA) that is a hormone-like substance known to repress Oct4 gene expression and induce cell differentiation is also used.

After 24 hours of preparation, the prepared screening device is added with various drugs respectively. The drugs include 2 mM VPA, 100 nM RA, and a mixture of 2 mM VPA and 100 nM RA. Each kind of drug is reacted with cells for 24 hours and then harvest cells. Next perform luciferase assay. The cells without the drug for 24 hours and treated by the drug for next 24 hours are called “24 hr cell”. In results of the luciferase assay, VPA exposure leads to higher level of Luciferase while RA decreases the level of Luciferase. Thus using luciferase gene, small-molecule compounds that induce pluripotency are expressed and found out.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A screening device that contains Nanog promoter, Sox2 promoter, Lin28 promoter, Oct4 promoter, and luciferase gene comprising:
a reporter plasmid that contains Nanog promoter, luciferase gene, and a G418 resistance vector being transfected to a host cell;
a reporter plasmid that contains Sox2 promoter, luciferase gene, and a G418 resistance vector being transfected to the host cell;
a reporter plasmid that contains Lin28 promoter, luciferase gene, and a G418 resistance vector being transfected to the host cell; and
a reporter plasmid that contains Oct4 promoter, luciferase gene, and a G418 resistance vector being transfected to the host cell;
wherein the screening device has different host cells comprising Nanog promoter, Sox2 promoter, Lin28 promoter, and Oct4 promoter individually

2. The device as claimed in claim 1, wherein the host cell is a somatic cell or a stem cell.

3. The device as claimed in claim 2, wherein the somatic cell is a myoblast.

4. The device as claimed in claim 2, wherein the stem cell is an embryonal carcinoma cell.

5. The device as claimed in claim 1, wherein the G418 resistance vector is a pStable vector.

6. A screening method of screen device that contains Nanog promoter, Sox2 promoter, Lin28 promoter, Oct4 promoter, and luciferase gene comprising the steps of:
constructing a screening device that contains Nanog promoter, Sox2 promoter, Lin28 promoter, Oct4 promoter, and luciferase gene;
adding an object under test into the screen device that contains Nanog promoter, Sox2 promoter, Lin28 promoter, Oct4 promoter, and luciferase gene; and
using a luminometer to detect and read luminescence of the screen device.
